(19) United States
(12) Patent Application Publication
(10) Pub. No.: US 2026/0053336 A1
(43) Pub. Date: Feb. 26, 2026

(54) MEDICAL DEVICES, SYSTEMS, AND METHODS

(71) Applicant: Boston Scientific Scimed, Inc., Maple Grove, MN (US)

(72) Inventors: Eric Noble LENT, Roberts, WI (US); Christine Ann RUDBERG, Greenwood, MN (US); Dennis LYNCH, Co. Cork (IE); Daniel Joseph GRASSO, Franklin, MA (US)

(73) Assignee: Boston Scientific Scimed, Inc., Maple Grove, MN (US)

(21) Appl. No.: 19/299,494

(22) Filed: Aug. 14, 2025

Related U.S. Application Data
(60) Provisional application No. 63/685,421, filed on Aug. 21, 2024.

(51) Int. Cl. A61B 1/00; A61B 1/005; A61B 1/307

(52) U.S. Cl. CPC A61B 1/00137; A61B 1/0011; A61B 1/0057; A61B 1/307

(57) ABSTRACT

Medical devices, systems, and related methods are discussed. The medical device may include a distal cap, e.g., the distal cap including an imaging device; an illuminator; a working channel; a first material enveloping at least a portion of each of the imaging device, the illuminator, and the working channel; and a second material enveloping the first material. The second material may be harder than the first material.

### CROSS-REFERENCE TO RELATED APPLICATIONS

This application claims the benefit of priority to U.S. Provisional Application No. 63/685,421, filed on Aug. 21, 2024, which is incorporated by reference herein in its entirety.

### TECHNICAL FIELD

Various aspects of this disclosure relate generally to medical devices and methods of use thereof. Furthermore, this disclosure relates to systems and methods for manufacture of medical devices, among other aspects.

### BACKGROUND

Often, portions of a medical device are created as a stand-alone injection molded component. For example, distal caps used in “camera in a catheter” medical devices, such as an endoscope or an ureteroscope, are often created as a stand-alone plastic injection molded component. After the distal cap is created, components such as a camera or working channel are typically inserted into the molded cap. This technique often causes the distal cap to be molded larger than necessary, in order to allow the camera and other components to be inserted into the molded distal cap and allow for some tolerance. This approach is not ideal and works against the desire to minimize the size of the distal cap, since the smaller distal cap size typically facilitates maneuvering through a body lumen without injuring the patient, among other benefits of the smaller cap size. Devices, systems, and methods of this disclosure may address some or all of these difficulties, and may address other problems in the art.

### SUMMARY

This disclosure relates to, among other things, medical devices, systems, and methods of use for medical devices, such as endoscopes or ureteroscopes, and methods of manufacturing such medical devices. Aspects of this disclosure may decrease the time to complete a procedure, such as removal of a kidney stone, from a patient's body, and may facilitate the maneuverability and other operation of the medical devices discussed herein. Further, for example, the methods of manufacture discussed herein may reduce the labor time to make a medical device, may allow for a more effectively sealed medical device, and may allow for the creation of a more durable medical device. Any of the systems, devices, and features thereof discussed herein may be used or adapted for use in any parts of the body, or be used in other ways. The medical devices and systems disclosed herein in various examples may include one or more of the features or components described in connection with any of the other examples.

According to some aspects, the medical device comprises a distal cap, the distal cap comprising an imaging device; an illuminator; a working channel; a first material enveloping at least a portion of each of the imaging device, the illuminator, and the working channel; and a second material enveloping the first material, wherein the second material is harder than the first material. The medical device may include one or more of the following features. The first material may be molded onto the imaging device, the illuminator, and the working channel via injection molding. The second material may be molded onto the first material via injection molding. The first material may completely cover by the second material. The distal cap may further comprise a light pipe distal to the illuminator. For example, the light pipe may include a plurality of branches extending to a side wall of the distal cap. The distal cap may further comprise a printed circuit board operably coupled to the imaging device and an illuminator, wherein the printed circuit board is enveloped by the first material. The distal cap may be coupled to a distal end of the shaft. A steering wire may extend through the shaft, and a distal end of the steering wire may be embedded in the first material and/or the second material of the distal cap. The second material may extend circumferentially around the imaging device, the illuminator, and the working channel. A first distal front face of the imaging device, a second distal front face of the illuminator, and a third distal front face of the working channel are not in contact with the first material. The first material and/or the second material may be transparent and/or translucent. The first material may include at least one first gate burr positioned proximal to the imaging device and the illuminator. The second material may include at least one second gate burr positioned at a proximal end of the second material. The second material may extend through a channel of the first material, and the second material may abut a distal front face of the illuminator.

The present disclosure also includes methods of manufacturing a distal cap for a medical device, e.g., a distal cap as discussed above or elsewhere herein. For example, the method may comprise positioning an imaging device, an illuminator, and a working channel in a mold; introducing in the mold a first material; introducing in the mold a second material different from the first material, wherein the second material sets to form the distal cap with the first material entirely enveloped by the second material; and removing the distal cap from the mold. The method may include one or more of the following features. The imaging device and the illuminator may be positioned within a cassette prior to positioning the imaging device, the illuminator, and the working channel in the mold. The method may further include stamping a distal end of a steering wire, wherein stamping the distal end changes a shape of the distal end; and positioning the distal end of the steering wire within the mold prior to introducing the first material to the mold. Positioning the imaging device, the illuminator, and the working channel in the mold may include positioning a plurality of imaging devices, a plurality of illuminators, and a plurality of working channels in the mold; and removing the distal cap from the mold may include removing a panel of distal caps from the mold.

Also included herein is a medical device comprising a shaft, and a distal cap coupled to a distal end of the shaft. The distal cap may comprise an imaging device; an illuminator; a working channel; a first material enveloping at least a portion of the imaging device, the illuminator, and the working channel, wherein the first material is opaque; and a second material enveloping the first material, wherein the second material is transparent and harder than the first material.

### BRIEF DESCRIPTION OF FIGURES

The accompanying drawings, which are incorporated in and constitute a part of this application, illustrate exemplary aspects of this disclosure and together with the description, serve to explain the principles of the present disclosure.

FIGS. 1A and 1B illustrate an exemplary medical device, in accordance with some aspects of this disclosure.

FIGS. 2A and 2B illustrate a side view and a front view of a distal cap of a medical device, in accordance with some aspects of this disclosure.

FIG. 3 illustrates a front view of a distal cap of a medical device, in accordance with some aspects of this disclosure.

FIGS. 4A and 4B illustrate a front view and a side view of a distal cap of a medical device, in accordance with some aspects of this disclosure.

FIGS. 5A and 5B illustrate a front view and a side view of a distal cap of a medical device, in accordance with some aspects of this disclosure.

FIGS. 6A, 6B, 6C, 6D, and 6E illustrate a process for manufacturing a portion of a medical device, in accordance with some aspects of this disclosure.

FIGS. 7A, 7B, and 7C illustrate a top view, a side view, and a second side view of various steps in manufacturing a distal cap of a medical device, in accordance with some aspects of this disclosure.

FIGS. 8A and 8B illustrate a top view and a side view of a step in manufacturing a distal cap of a medical device, in accordance with some aspects of this disclosure.

FIG. 9 illustrates a side view of a pickout bar being used to manufacture a distal cap of a medical device, in accordance with some aspects of this disclosure.

FIG. 10 illustrates a side view of a molded distal cap of a medical device, in accordance with some aspects of this disclosure.

FIGS. 11 and 12 illustrate a side view and a front view, respectively, of a mold used to manufacture a distal cap of a medical device, in accordance with some aspects of this disclosure.

FIG. 13 illustrates a front view of a distal cap of a medical device, in accordance with some aspects of this disclosure.

FIGS. 14A, 14B, and 14C illustrate various steps in manufacturing a distal cap of a medical device using a mold system, in accordance with some aspects of this disclosure.

FIGS. 15A and 15B illustrate a panelized circuit at two different points in the manufacturing process of a plurality of portions of a medical device, in accordance with some aspects of this disclosure.

FIGS. 16 and 17 illustrate two different examples of a distal cap for a medical device, in accordance with some aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” Relative terms such as, for example, “about,” “substantially,” “approximately,” etc., are used to indicate a possible variation of ±10% in a stated numeric value or range. The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures.

Aspects of various devices and methods of manufacture are now described. Some aspects are described with reference to noninvasive procedures, such as urology or endoscopy procedures, wherein a sheath is advanced to a treatment site, and a distal cap of the sheath is used to treat a patient at a treatment site. In urology procedures, for example, the sheath may be inserted into the urethra, moved through the bladder and ureter, and advanced into a calyx of a kidney; and the capture device may be extended distally from the sheath and/or a working channel of a ureteroscope or other medical device to engage one or more stones and/or stone fragments located in the calyx.

References to a particular type of medical procedure, such as a urology procedure; capture device, such as a basket or grasper; organ, such as a kidney; and/or object, such as a stone or stone fragment, are provided for convenience and not intended to limit this disclosure. Accordingly, the concepts described herein may be utilized for any analogous medical device or system.

The medical devices, systems, and methods discussed herein may facilitate manufacture of a medical device, may provide a more durable medical device as a result of the manufacturing techniques described herein, may more effectively seal components of a medical device, may create more durable medical devices compared to other manufacturing techniques, and/or may reduce labor time and effort required during assembly of medical devices, among other benefits.

Although ureteroscopes are referenced herein for illustration purposes, it will be appreciated that the disclosure encompasses any suitable medical device configured to allow an operator to access, and sometimes view, internal body anatomy of a subject (e.g., patient) and/or to deliver medical instruments, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein, and any of the manufacturing processes described herein may be used to manufacture any of these medical devices.

According to some aspects of this disclosure, the medical device may include a handle coupled to a shaft, e.g., wherein the shaft may extend to a distal articulating portion. The handle may include one or more actuators to actuate movement of the distal articulating portion. The distal articulating portion may include one or more cameras, one or more illuminators (e.g. LED lights, etc.), and/or one or more elevator assemblies, and one or more working channels may extend through the shaft to distal articulating portion. Any of the components of the distal articulating portion, such as one or more cameras, one or more illuminators, one or more elevator assemblies, and one or more working channels, may be incorporated into a distal cap of the shaft, which may be part of the distal articulating portion.

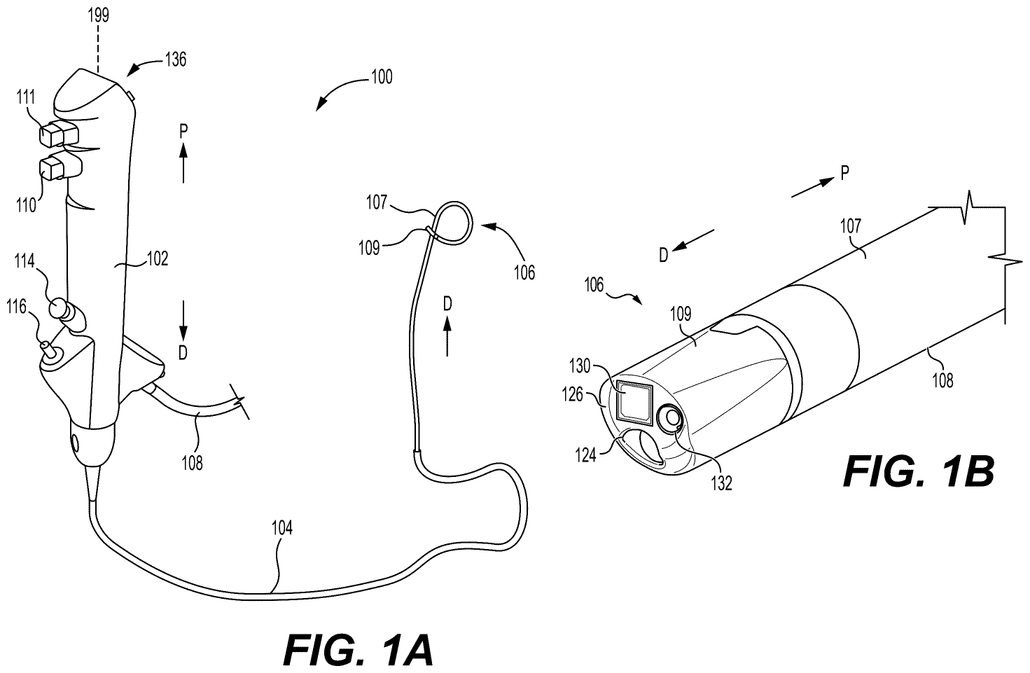

FIG. 1A illustrates an exemplary medical device 100, or insertion device, including a handle 102 and a shaft 107 extending distally from handle 102. Medical device 100 may have a central longitudinal axis 199. Shaft 107 may include a distal articulating portion 106 including an articulation section of the shaft 107 with a distal cap 109. In some examples (as shown in FIG. 1A), medical device 100 may include an over sheath 104 positioned over a portion of shaft 107; in other examples medical device 100 does not include an over sheath 104. Handle 102 may include one or more actuators 110, 111, 136, one or more fluid ports 114, and one or more working channel ports 116. An umbilicus 108 may be connected to handle 102, and umbilicus may electronically connect components of medical device 100 to a control unit or other electronic device. In some examples, umbilicus 108 may also supply fluid to one or more fluid channels of medical device 100 and/or suction (aspiration) to one or more channels of medical device 100. In some examples, handle 102 may communicate wirelessly with a control unit or other device.

Distal articulating portion 106 may include one or more cameras and one or more illuminators. One or more actuators, e.g., actuator 136, of handle 102 may control the one or more cameras and/or one or more illuminators of distal articulating portion 106. One or more actuators 110, 111, 136 may be or include push-button actuators, knobs, or any other type of suitable actuator, such as an electronic screen touch interface. For example, actuators 110, 110 may be push-button actuators and may be configured to control articulation (e.g. bending) of distal articulating portion 106 of shaft 107.

Actuators 110, 111 may control the movement of articulation wires extending through shaft 107 to distal articulating portion 106; and articulation wires may be used to move (e.g. bend) distal articulating portion 106, for example to adjust the position of distal cap 109.

FIG. 1B illustrates a magnified perspective view of distal articulating portion 106 of medical device 100, including distal cap 109 and shaft 107. Distal cap 109 may be coupled to a distalmost end of shaft 107, may be cylindrical or any other suitable shape, and may include a distal front face 126. An imaging device 130 (e.g. camera, etc.), an illuminator 132 (e.g. a light emitting diode (LED), etc.), and an opening 124 of a working channel may be positioned at the distal front face 126 of distal cap 109. In other examples, distal cap 109 may include one or more side-facing imaging devices and/or one or more side-facing illuminators and/or one or more side-facing working channel openings positioned at a side wall of distal cap 109. In some examples, distal cap 109 may include one or more sensors, such as a temperature sensor or pressure sensor (or a sensor configured to measure both temperature and pressure, for example). Distal cap 109 is shown in FIG. 1B with beveled side walls, however in other examples, distal cap 109 may include cylindrical (e.g. curved) side walls or any other shape sidewall(s). A working channel may extend longitudinally through shaft 107 from working channel port 116 of handle 102 through shaft 107 to working channel opening 124 of distal cap 109.

Distal cap 109 may be an injection molded component. The devices and methods described hereinbelow relate to injection molding of distal cap 109 around electronic and other components, such as imaging device 130, illuminator 132, and a working channel. By manufacturing distal cap 109 via injection molding around the components within distal cap 109, the size of distal cap 109 may be minimized; the components may be encapsulated, sealed, and protected from fluids and other material; the working channel may be incorporated into the device with strong structural support at the distal end; articulation wires may be encompassed by distal cap 109 and provide a structurally capable means to support steering forces; the distal cap may include termination tips or other articulation joint materials to allow a more seamless, continuous shaft; and/or the labor invested to manufacture the medical device may be reduced; among other possible benefits.

According to some aspects, the injection molding process involves melting medical-grade, e.g., biocompatible, material such as plastics or other polymers (or other suitable materials known in the art) and molding them into the desired shape of medical device component(s). Specifically, the process may include melting plastic resin pellets and injecting the molten material into a mold to create a desired shape. This process may allow for precise control over the component's size and shape, e.g., within applicable quality and regulatory standards. The material used in injection molding may include thermoplastics, thermosets, and/or any other suitable material.

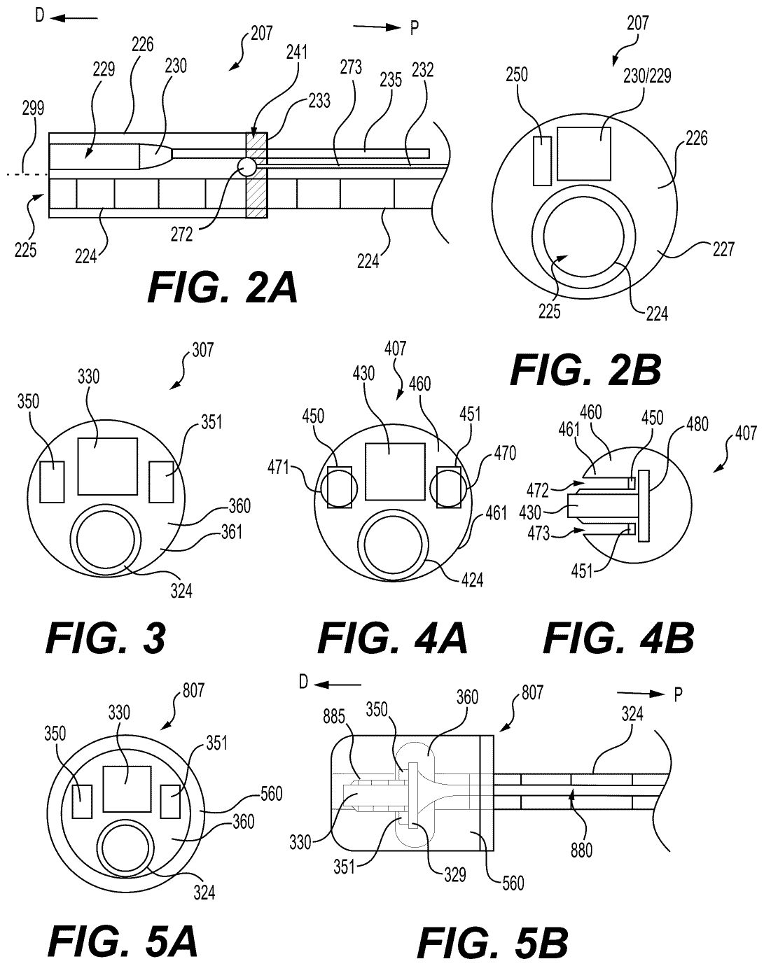

FIGS. 2A and 2B illustrate side and front views, respectively, of an exemplary distal cap 207 prepared using injection molding. To mold distal cap 207, an imaging device 230, a working channel 224, an illuminator 250, and an articulation wire 232 may be placed into a fixture and a material injection molded to form distal cap 207 around each component. In some examples, a proximal end 233 of distal cap 207 may include a rough or irregular surface 241 to facilitate coupling distal cap 207 to a shaft of a medical device, such as shaft 107 in FIGS. 1A and 1B. Electrical cable(s) 235 may extend proximally from proximal end 233, and may be configured to connect imaging device 230 and illuminator 250 with a proximal portion of the medical device, such as connection with a printed circuit board (PCB) positioned within handle 102. As shown in FIG. 2B, the body 226 of distal cap 207 may extend entirely around each radially-outer facing surface, relative to a central longitudinal axis 299 of distal cap 207, of each of the imaging device 230, the illuminator 250, and the working channel 224.

In some examples, a distal portion of electrical cable(s) 235 may be enveloped by body 226 of distal cap 207; in other examples, electrical cable(s) 235 may be positioned entirely outside of body 226. In some examples, a distal opening 225 of working channel 224 may be positioned at a distal front face 227 of body 226, such that opening 225 is exposed to the area exterior to distal cap 207. A distalmost end 272 of articulation wire 232 may be circular and may be larger relative to a proximal portion of the articulation wire 232. For example, distalmost end 272 may be spherical and may have a diameter larger than the diameter of the longitudinal shaft portion 273 of articulation wire 232. Distalmost end 272 may be entirely enveloped within body 226, and may facilitate coupling articulation wire(s) 232 to body 226. Articulation wire(s) 232 may extend proximally to a handle of the medical device (e.g., handle 102 of medical device 100) and be coupled to one or more actuators to articulate a distal portion of the medical device, including distal cap 207.

In some examples, one or more of the imaging device 230, illuminator 250, and/or working channel 224 may be entirely enveloped by body 226 of distal cap 207, with all exterior surfaces covered by and/or bonded to body 226 (e.g., other than surfaces used in imaging and/or illumination during a medical procedure). By entirely enveloping components of distal cap 207 with body 226, distal cap 207 may more effectively seal components and prevent fluids from damaging components of distal cap 207.

A light pipe 229 may be positioned in front of imaging device 230. Light pipe 229 may be transparent or translucent, and may be configured to receive light from exterior of distal cap 207 and direct the light towards imaging device 230. Light pipe 229 may be cylindrical, rectangular (as shown in FIG. 2B), or any other suitable shape. Light pipe 229 may be configured to transmit light from an illuminator to a distal end of the light pipe 229, such as to the exterior of distal cap 207. Light pipe 229 may also be configured to receive light from exterior of distal cap 207 and transmit the received light to imaging device 230.

FIG. 3 illustrates a front view of a first portion 307 of another exemplary distal cap (see, e.g., distal cap 807 in FIGS. 5A-5B). FIG. 3 illustrates a first step in manufacturing the distal cap in which imaging device 330, circuit board 329 (shown in FIG. 5B), illuminators 350, 351, and working channel 324 are injection molded into a first portion 307, with a first material 360 coupling each component together. First material 360 may have a distal front face 361, and imaging device 330 may extend to the distal front face 361. Circuit board 329 may be coupled to imaging device 330 (e.g. camera, etc.), illuminators 350, 351, and one or more electrical cables. First material 360 may be a material that does not deform working channel 324 but still bonds to it, supports it, and maintains working channel 324 in the proper position relative to imaging device 330, circuit board 329, and illuminators 350, 351. First material 360 may be a thermally conductive material configured to remove heat from electronic components of the distal cap, such as from the imaging device 330 and/or illuminators 350, 351. In some examples, first material 360 may be configured to block light to prevent light noise or stray light in the imaging device 330, and may be dark in color and optically opaque. In examples in which first material is opaque, first material 360 may provide openings for a portion of each of the illuminators 350, 351 to prevent blockage of light from illuminators 350, 351. First material 360 may be elastic and may be configured to absorb shock from movement of the distal cap and/or shock generated during a medical procedure (e.g., by a laser breaking apart a kidney stone). For example, elasticity of first material 360 may allow imaging device 330 and/or illuminators 350, 351 to move when the distal cap impacts an object, thus potentially preventing damage to imaging device 330 and/or illuminators 350, 351.

FIGS. 4A and 4B illustrate a front and side view of a first portion 407 of another exemplary distal cap. FIGS. 4A and 4B illustrate a first step in manufacturing a distal cap, similar to the distal cap of FIG. 3 and distal cap 807 discussed below, in which imaging device 430, circuit board 480, illuminators 450, 451, and working channel 424 are injection molded into first portion 407, with a first material 460 coupling each component together. First material 460 may have any of the attributes of first material 360. A first channel 472 may be formed in the body 461 of first portion 407 in front of one of the illuminators, e.g., first illuminator 450; and a second channel 473 may be formed in the body 461 of first portion 407 in front of the other of the illuminators, e.g., second illuminator 451. Each of first channel 472 and second channel 473 may be formed by a fixture, such as a molding fixture, used to prevent injection molding material from covering illuminators 450, 451; thus forming respective channels 472, 473 that extend between each illuminator 450, 451, respectively, to an exterior of first portion 407 (as shown in FIG. 4B).

In some examples, the fixtures used to form channels 472, 473 may be light pipes, such as a cylindrical body configured to transfer light from illuminators 450, 451 to an exterior portion of first portion 407 (e.g., exterior to the distal tip). In other examples, the fixtures used to form channels 472, 473 may be part of a mold used to form first material 460; and then a second material (such as second material 560 shown in FIGS. 5A and 5B, which will be discussed in detail below) may fill each channel 472, 473. In such an example, the second material 560 may be transparent, translucent, or otherwise configured to transmit light such that light generated by illuminators 450, 451 may reach an exterior of the distal cap. In some examples, channels 472, 473 may be directed towards a field of view of the imaging device 430 to provide illumination to the field of view of the imaging device 430.

FIGS. 5A and 5B illustrate front and side views, respectively, of distal cap 807 formed of first material 360 and including components of the distal cap discussed in FIG. 3, and a second material 560. Although distal cap 807 is shown incorporating first material 360 shown in FIG. 3, distal cap 807 may alternatively incorporate first material 460 from FIGS. 4A and 4B (e.g., in combination with electronic and/or other components of FIG. 3 and/or FIGS. 4A-4B). After forming first material 360 around imaging device 330, circuit board 329, illuminators 350, 351, and working channel 324, second material 560 is injection molded over the first material 360. Second material 560 may be harder than the first material 360, and may be biocompatible. In some examples, second material 560 may be transparent or translucent, and/or may provide a space such that a front lens of the imaging device 330 is uncovered and/or may provide spaces such that a portion of each illuminator 350, 351 is uncovered. In some examples, second material 560 may be or comprise polycarbonate, and/or may be or comprise polypropylene. Second material 560 may be configured to dissipate heat. Second material 560 may seal components of distal cap 807 from fluids, may increase the stability and strength of the distal cap 807, may support one or more articulation wires, and/or may facilitate coupling distal cap 807 to a shaft of a medical device (e.g., shaft 107 of medical device 100).

Distal cap 807 may be formed of an undermold of first material 360 and an overmold of second material 560. The undermold of first material 360 may encapsulate sensitive or weaker components via a low-pressure molding process while bonding them together in proper position. A low-pressure undermold approach may mitigate the risk of damaging or deforming the components as the undermolded body may encapsulate and protect these components from potentially higher-pressure/temperature materials which may be used during the subsequent overmolding process. After the second step of overmolding the second material 560 over the first material 360, the distal cap 807 may be ready for assembly onto the medical device shaft (e.g., shaft 107).

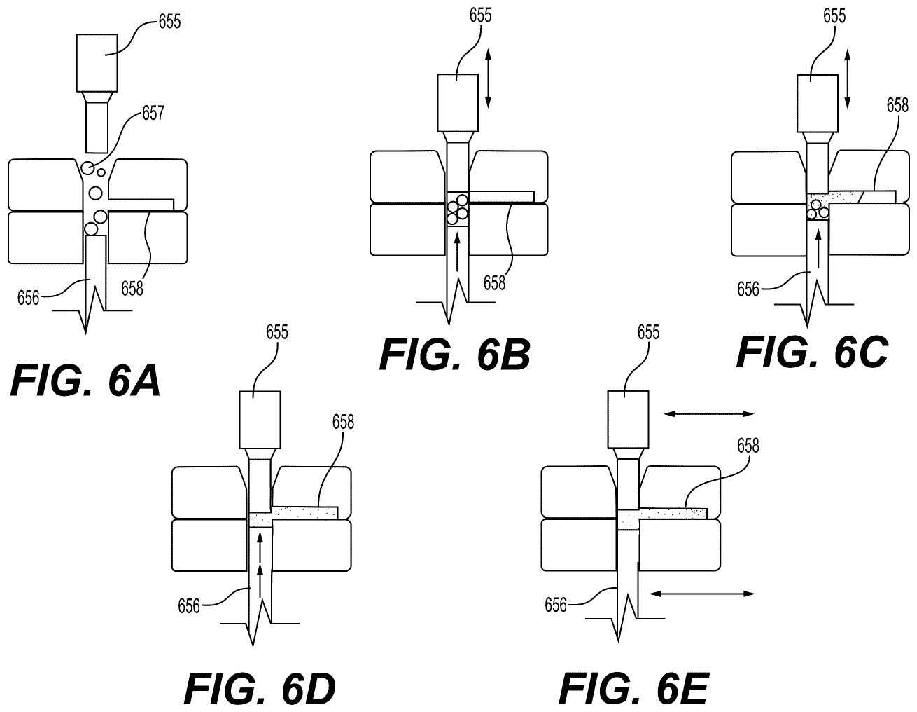

Ultrasonic (Injection) Molding (USM) may be used for either or both the undermolding step (e.g. forming of first material 360) or overmolding step (e.g. forming of second material 560). USM uses an ultrasonic horn 655 (“sonotrode”) to act on and melt a relatively small measured amount of polymer granules 657 using ultrasonic energy. The resultant molten resin may be sufficient for a single shot (injection). A secondary piston 656 then injects the molten resin directly into the mold cavity 658 to encapsulate the electronic components to create the undermold (e.g., first material 360), or additionally or alternatively may inject resin around the undermolded insert, thereby creating the final overmolded component. FIGS. 6A-6E illustrate an exemplary process of USM, which may be used to form a distal cap, such as distal cap 807.

The type of material, e.g., polymer, used to mold a distal cap 109, 207, 807 may be selected depending on the type(s) of electronic components to be encapsulated, for example due to the temperature and pressures that the electronic components typically withstand. A thermoplastic, such as Loctite® PA 6682, is a clear polyamide (nylon) and may be suitable within a low-pressure injection molding system. A thermosetting resin, such as a low temperature silicone (e.g. LSR Select™ from Elkem) may be suitable to encapsulate electronic components capable of higher temperature resistance, or such a thermosetting resin could be used for the overmolding (e.g. second material 560).

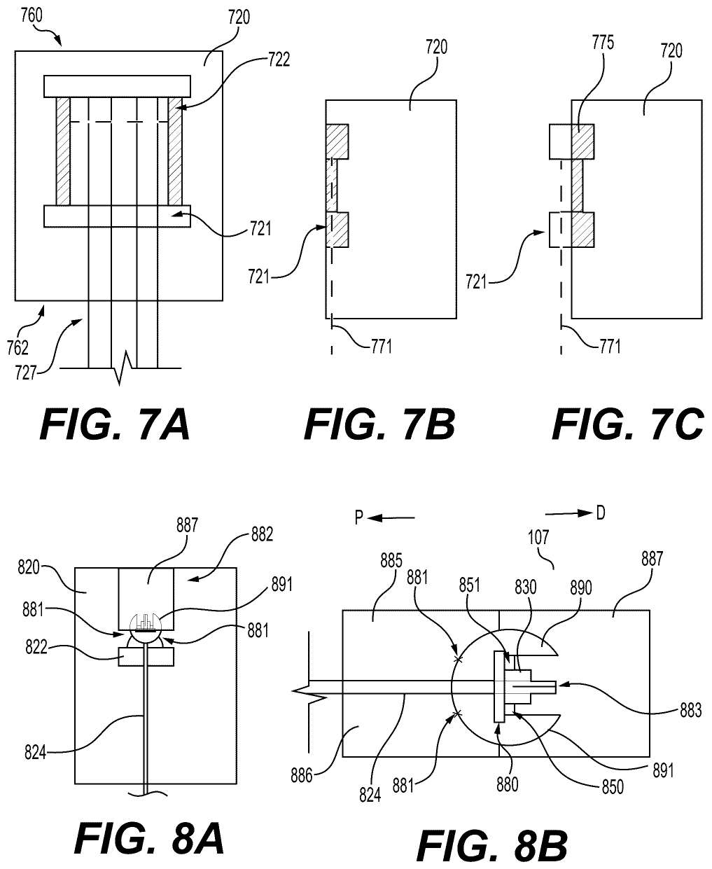

FIG. 7A illustrates a front view of a mold 720 with a cassette 721 that may be used for molding a distal cap such as distal cap 109, 207, 807, or component thereof. The top 760 of mold 720 and bottom 762 of mold 720 are shown in FIG. 7A to show the orientation of mold 720 relative to the parts 727 loaded into mold 720. Cassette 721 may facilitate loading and unloading components for molding a distal cap 109, 207, 807. Cassette 721 may be utilized for molding both a first material 360 portion (undermold) and a second material 560 portion (overmold). Parts 727 may be positioned in cassette 721 for molding multiple distal caps 109, 207, 807 at the same time, which could be used in a mass production environment. Parts 727 for four separate distal caps 109, 207, 807 are shown positioned within mold 720 in FIG. 7A. The cavity block 722 of mold 720 is shown in FIG. 7A, and cavity block 722 may be configured to receive material to be molded into a portion of distal cap 109, 207, 807.

FIG. 7B illustrates parts 771 loaded into cassette 721 of mold 720 before material is applied to parts 771. FIG. 7C illustrates parts 771 after material has been applied via the mold 720, and the molded parts 771 are ready to be unloaded from mold 720. Mold 720 may be a modular design that utilizes interchangeable mold cavities and pickout bars. Pickout bars (such as pickout bar 887 shown in FIG. 8B) may be used for part 771 loading/unloading into mold 720, and mold runners and gates may be designed into the pickout bars and/or the cavity inserts of a mold 720.

Loading and unloading of a medical device assembly, such as parts including an image sensor, illuminators, and working channel, may be guided by an alignment pin 883 (shown in FIG. 8B). To accomplish this, the molding operator may place a cassette 721 on a flat surface to load the part 771. This step may include loading the working channel (of part 771) onto the alignment pin 883. In some examples, this step may be fully automated for ease of assembly. Next, the cassette 721 may be loaded into the mold 720 via either manual or automated means. Once the cassette 721 is properly loaded, the molding machine may close the mold 720 and begin the molding cycle. The molding cycle may apply the material to parts 771 to form a distal cap 109, 207, 807. Once the mold 720 opens, the cassette 721 may be partially ejected to enable an operator (e.g., a manual operator or robot) to remove the cassette 721 from the mold 720.

At this step in the process, the design of the pickout bar 887 may form the distal end of the molded body 890. FIGS. 8A and 8B illustrate a working channel 824, printed circuit board 880, imaging device 830, and illuminators 850, 851 positioned within a mold 820, and a pickout bar 887 is positioned over these components with pin 883 positioned within working channel 824. In FIG. 8B, a top portion 885 of mold 820 and a bottom portion 886 of mold 820 is shown. A cavity 891 is formed between mold 820 and pickout bar 887, and injection of material into cavity 891 forms the molded body 890. A recess 882 in mold 820 may be configured to receive the pickout bar 887. Material is injected into cavity 891 through gates 881.

Pickout bar 887, as shown in FIG. 8B, may be configured to form channels or tunnels in which light generated by illuminators 850, 851 may pass through. Additionally, this shape of pickout bar 887 may allow molten plastic (or other material used in molding) to form around the working channel 824 and printed circuit board 880 to both support the components and create a watertight seal. Features such as alignment pin 883 of pickout bar 887 may guide components into proper placement, e.g., to ensure component placement when forming a distal cap 109, 207, 807. Any technique known in the art may be used within the mold design to control alignment pins 883. For example, a hydraulic cylinder or servo motor may control actuators for the alignment pin(s) 883, which may protrude from either half of the mold 820 to support the electronic components within the mold 820 (e.g. fixed and moving half of mold 820). Alignment pin(s) 883 may be retracted to the mold 820 cavity surface shortly after injection of the molten material starts or at the start of the holding (packing) phase of the injection molding cycle, when the resin is still in a semi-molten state to backfill a recess created with molten resin so that the alignment pin 883 witness holes are no longer visible on the molded part. The retraction of alignment pin(s) 883 may be a step in the manufacturing process, and may be facilitated by a motor or other mechanism to retract the alignment pin(s) 883. The electronic components may become fully encapsulated and no area of them are exposed to the outer surface of the final molded part (e.g. distal cap 109, 207, 807).

FIG. 8A illustrates a gate block 822 and gates 881 of mold 820 positioned at a proximal end of the molded body 890 (e.g. first material 460 part, etc.). By positioning gates 881 at a proximal portion/end of molded body 890, gate burrs formed in molded part 890 may be fully covered during the overmold process, e.g., eliminating the potential for exposed gate burrs to harm a patient.

Alignment pin 883 of pickout bar 887 may be configured to support working channel 824 during the molding process. Alignment pin 883 may be configured to keep the working channel 824 open during the entire process, to protect against possible damage, deformation, or closing due to mold pressures and temperatures. Alignment pin 883 may facilitate part placement, and also may facilitate positioning of working channel 824 relative to the other components during the loading of components into mold 820. Alignment pin 883 may have features such as draft and draw polishing to prevent damage and to aid in the removal of the working channel 824 from the pickout bar 887 after molding.

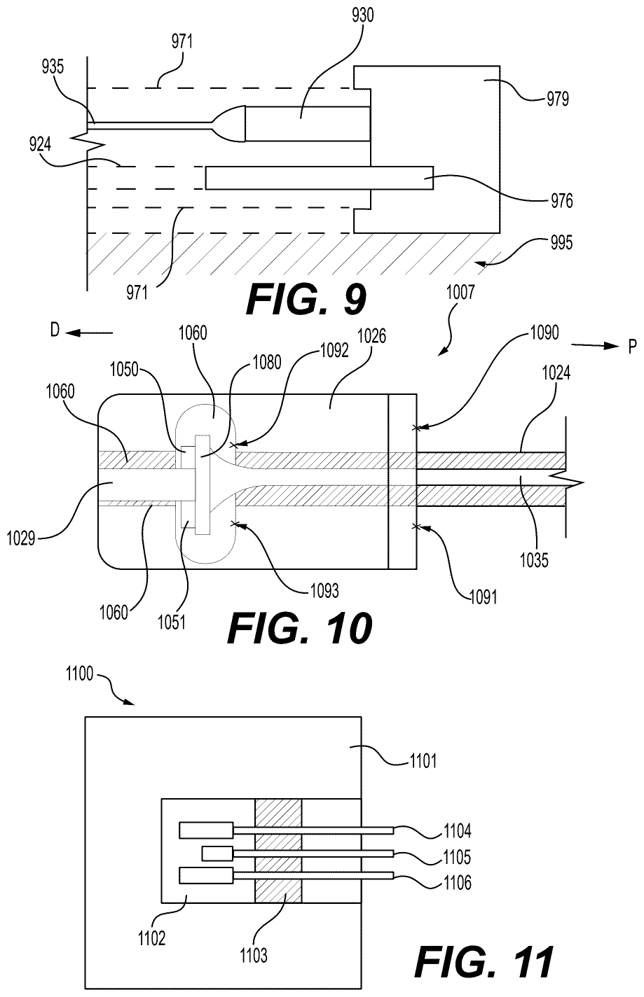

FIG. 9 illustrates a side view of another exemplary pickout bar 979 with a pin 976 configured to align with a working channel 924, for example of a catheter 971 (or shaft of a medical device). An imaging device 930 is shown positioned flush with pickout bar 979, and cable 935 extends proximally from imaging device 930. Pickout bar 979 is shown resting on a surface 995, and may be positioned over catheter 971 on a surface 995 prior to molding. Pin 976 may be a steel pin, and may be removable from the pickout bar 979.

FIG. 10 illustrates a side view of a distal cap 1007 including a printed circuit board 1080, an imaging device 1029, illuminators 1050, 1051, a cable 1035, and a working channel 1024. A first material 1060 (e.g., undermold) is shown coupled to the printed circuit board 1080, illuminators 1050, 1051, imaging device 1029, and working channel 1024; and a second material 1026 (e.g., overmold) is shown molded over first material 1060. During manufacture of distal cap 1007, molten material (e.g., molten plastic) may enter the mold cavity through a mold gate or multiple mold gates. As briefly discussed above, strategic placement of gates 1092, 1093 may minimize gate appearance, mechanical interference, and/or the presence of external burrs that could harm the patient. Additionally, the number of gates 1092, 1093 may aid in material processing as the gates 1092, 1093 moderate and direct the material entering the mold cavity. With regard to the first material 1060 (e.g., the undermold), double gates 1092, 1093 may be placed on the proximal end of the distal cap 1007. This placement may ensure any gate burrs would be completely covered during the overmold process (e.g. application of second material 1026), eliminating the potential for gate burrs to harm the patient. Dual gates 1090, 1091 for the second material (e.g., overmold) may be placed on a feature that does not directly interact with the patient, such as the proximal end of the overmolded body, as shown in FIG. 10.

Any of the mold designs described herein may be used in a horizontal molding machine, e.g., encasing the full assembly within molding safety guards. This may prevent damage to the portion of the medical device assembly (e.g., shaft assembly or catheter assembly) which is outside the mold during the molding process. A fully automated molding cell may be used to provide consistent loading/unloading of components. Also, any of the mold designs described herein may be used in a vertical molding machine and may include adaptations to enable vertical molding.

FIG. 11 illustrates a side view of another exemplary mold 1100 including a mold base 1101, a cavity insert 1102, and a pinch-off 1103. Pinch-off 1103 in the mold 1100 may form a mechanical seal between the mold 1100 and cables/wires 1104, 1105, 1106 extending from components of a distal cap. Pinch-off 1103 may prevent the molten material (e.g., molten polymer) exiting the mold 1100 at the gap(s) between the mold 1100 and cable(s)/wire(s) 1104, 1105, 1106. Pinch-off 1103 may be manufactured from a suitable metal or metal alloy such as steel or aluminum, or a high strength polymer such as Delrin®, Nylon, or PEEK. In this configuration, a medium to high hardness resilient silicone pinch-off could be placed in the mold in order to conform to irregular cable/wire geometry. Pinch-off 1103 may be cylindrical, rectangular, or any other suitable shape; and may include channels to receive cable/wires 1104, 1105, 1106 and/or other components.

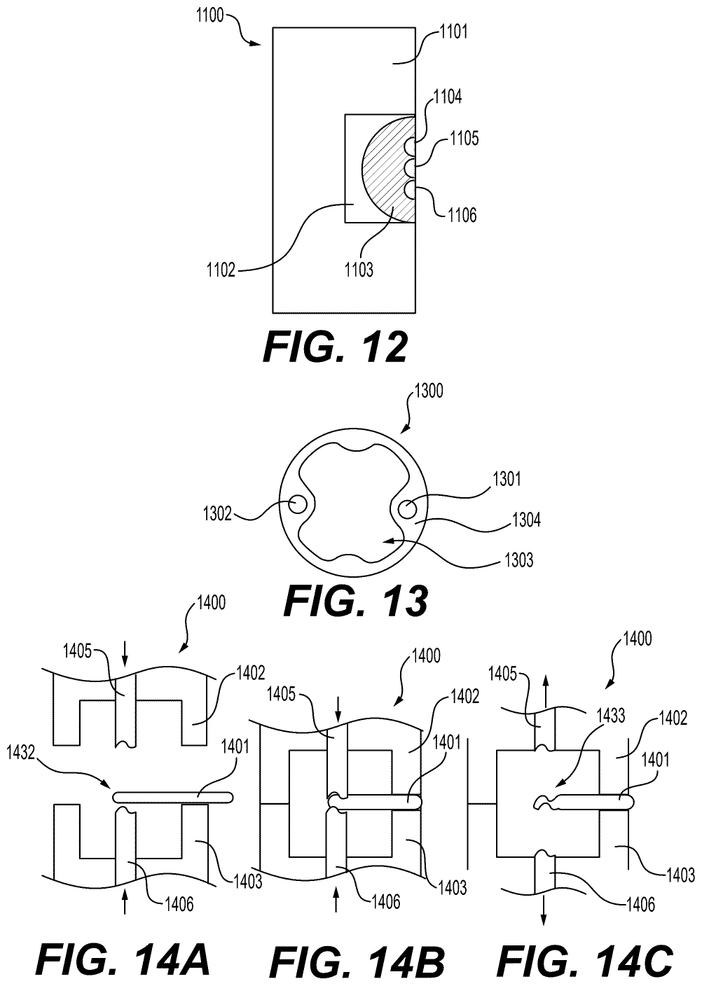

FIG. 12 illustrates a front view of mold 1100 including mold base 1101, mold cavity insert 1102, pinch-off 1103, and cable/wires 1104, 1105, 1106. Pinch-off 1103 may be removable from mold base 1101, and pinch-off 1103 may have any number of channels/recesses to receive corresponding components of a distal cap.

FIG. 13 illustrates a rear view of another exemplary distal cap 1300 including a body 1304, a cavity 1303 for components of distal cap 1300 (e.g., imaging device, illuminators, etc.), and lumens/recesses 1301, 1302. Each lumen/recess 1301, 1302 is configured to receive an articulation wire, such that the articulation wire(s) is/are coupled to distal cap 1300. Articulation/steering wires may be placed inside the length of a medical device (e.g., inside the shaft and the handle) to articulate or steer the distal portion (e.g., distal articulating portion of the shaft) by movement of an actuator in the handle. For example, two articulation wires may be used to provide steering along one plane, and four articulation wires may be used for four-way steering, e.g., along two orthogonal planes. The injection molding process described herein may allow final mechanical anchoring and fixing of the articulation/steering wires, and may also assist in minimizing space consumed within the distal cap by the articulation/steering wires. This anchoring may take place during the formation of first material 360, 460, 1060 (e.g. the undermold process); and may also take place or be further enhanced during the formation of second material 560, 1026 (e.g. the overmold process). In some examples, the strength of the adhesive bonds around the articulation/steering wire may be sufficient to hold without ends or ferrules to “grab onto,” for example with an articulation wire without an expanded end.

In some examples, articulation/steering wire(s) may include “T” ferrules or another bent feature at a distalmost end of the articulation/steering wire(s), and the ferrules or other bent feature may facilitate holding the articulation/steering wire(s) in place within a mold, such as within first material 360, 460, 1060 and/or second material 560, 1026. By fixedly coupling the distal end of the articulation/steering wire(s) to first material 360, 460, 1060 and/or second material 560, a user may pull the articulation/steering wire(s) to pull the distal cap 109, 207, 807, 1007, 1300.

FIGS. 14A, 14B, and 14C illustrate an exemplary manufacturing process to secure a distal end 1433 of an articulation/steering wire 1401 in manufacture of a distal cap. Straight ends 1432 of the steering wires 1401 may be placed on slotted forming pins 1405, 1406 of a stamping/forming system 1400 that stamp a defined shape on the end of the wire as the mold 1402, 1403 closes. These forming pins 1405, 1406 may be coupled to hydraulic or servo driven actuators, and may be withdrawn to the mold 1402, 1403 surface at the start of molten resin injection (FIG. 14C). This may allow full encapsulation of the steering wire distal end 1433, with the added benefit of a retaining feature (e.g., the “S” shape or other irregular shape of distal end 1433) embedded within the molded component, e.g., improving steering wire 1401 anchoring. For example, the “S” shaped distal end 1433 shown in FIG. 14C may provide a more resilient anchoring of steering wires 1401 compared to a straight distal end 1432 shown in FIG. 14A.

Generally, low-pressure overmolding technology may be less precise compared to traditional injection molding, mainly due to higher viscosity of the melt and lower possible packing pressures during low-pressure overmolding. In order to circumvent the lower precision, the overmolded material (e.g., plastic) may be injected over a larger area and diced into individual parts later. This technique also may be advantageous in higher volume manufacturing settings, where the electrical components may be panelized. In such an example, the entire panel may be placed into a mold, thus encapsulating a series of parts in a single shot of molten material. The final component may be achieved through a milling machine, laser etch, water jet or any other subtractive manufacturing process.

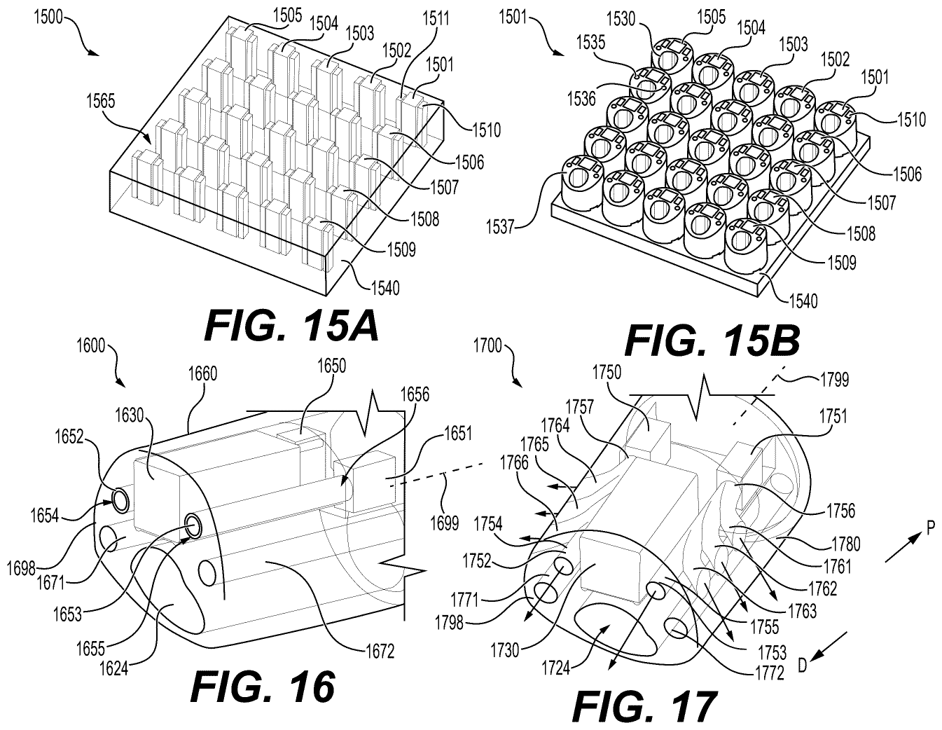

FIG. 15A illustrates a perspective view of panelized electronic components 1500 (e.g. imaging devices 1501-1509 and illuminators 1510, 1511) after injection molding a first material 1565 over the panelized electronic components 1500. After first material 1565 is applied to the panelized electronic components 1500, the panelized electronic components 1500 may be cut into a plurality of distal caps (e.g. 1535, 1536) via a milling machine, laser etching, water jet and/or any other subtractive manufacturing process. FIG. 15B illustrates electronic components panel 1501 after first material 1565 is applied via injection molding and individual distal caps 1535, 1536 have been cut out of the panelized electronic components 1500. Note the distal caps 1535, 1536 are still connected via a panel 1540 after the initial forming of distal caps 1535, 1536; and may be further processed and cut into separate distal caps 1535, 1536. FIG. 15B illustrates a molded panel 1501 with twenty-five distal caps 1535, 1536 cut out of the molded panel 1501. The distal front face 1537 of each distal cap 1535, 1536 may include a working channel opening 1530.

In other examples, distal cap 109, 207, 807 may be manufactured using three-dimensional (3D) printing in which material is printed around electronic components of distal cap 109, 207, 807 to embed the electronic components into the material (e.g., plastic or other printed material). The process of 3D printing distal caps 109, 207, 807 may include accurate positioning of electronic components on a build plate prior to 3D printing the distal caps 109, 207, 807, allocating a cavity in the part structure, and moving a nozzle of a 3D printing machine around the electronic/electrical components without interfering with those components.

In some examples, distal caps 109, 207, 807 may be formed using silicone. For example, first material 360, 460, 1060 (e.g., the undermold process) and/or second material 560, 1026 described herein may be or comprise silicone. Room temperature vulcanizing (RTV) silicone cures at room temperature, and medical grades are available in various durometers. Although the available durometers are generally softer than rigid plastics like polycarbonate, a silicone distal cap 109, 207, 807 may be desirable to improve patient comfort, e.g., for a less traumatic patient experience. The distal caps may have other stronger mechanical components embedded in the silicone, e.g., to serve as an anchor for components that experience higher forces, like steering wires.

FIG. 16 illustrates an exemplary distal cap 1600 including an imaging device 1630, illuminators 1650, 1651, a working channel 1624, exterior surface 1660, and steering wires 1671, 1672. Steering wires 1671, 1672 may extend longitudinally substantially parallel to a central longitudinal axis 1699 of distal cap 1600. A light pipe and/or optical fiber 1652, 1653 may extend from each illuminator 1650, 1651, respectively, to a distal front face 1698 of distal cap 1600. Light pipe and/or optical fiber 1652 may be configured to transfer light from illuminator 1650 to an area adjacent distal front face 1698, and a proximal end of light pipe 1652 may abut illuminator 1650 and a distal end 1654 of light pipe 1652 may be at the distal front face 1654 of distal cap 1600.

Light pipe and/or optical fiber 1632 may be configured to transfer light from illuminator 1651 to an area in front of distal front face 1698, and a proximal end 1656 of light pipe 1653 may abut illuminator 1651 and a distal end 1655 of light pipe 1653 may be at the distal front face 1654 of distal cap 1600. As shown in FIG. 16, light pipes 1652, 1653 may be positioned on opposite sides of imaging device 1630, and may extend substantially parallel to each other. Light pipes 1652, 1653 may be cylindrical, rectangular, another polygonal shape, or any other suitable shape. Light pipes 1652, 1653 may be encapsulated by an opaque material (e.g., opaque plastic or other material) in the same manner as the electronic components. Overmolding the light pipes 1652, 1653 may allow for placement of bigger and stronger illuminators (e.g. LEDs) proximally from the imaging device 1630; or placement on the same plane as the imaging device 1630, e.g., for ease of panelization during manufacturing. In the latter case, mounting the imaging device 1630 and illuminator(s) 1650, 1651 on the same plane may be a concern if the illuminators 1650, 1651 are much shorter than the imaging device 1630 (that the LEDs light output is diminished). By using light pipes 1652, 1653, light produced from illuminators 1650, 1651 may be transferred to an area in front of and proximate to the distal end of imaging device 1630.

FIG. 17 illustrates another exemplary distal cap 1700 including an imaging device 1730, a working channel 1724, illuminators 1750, 1751, steering wires 1771, 1772, exterior surface 1780, light pipes 1752, 1753, and a central longitudinal axis 1799. Light pipe 1752 may include a proximal end 1757 coupled to or adjacent to illuminator 1750, three branches 1764, 1765, 1766 curved towards a side surface of distal cap 1700, and a distal end 1754 at a distal front face 1798 of distal cap 1700. Light pipe 1753 may include a proximal end 1756 coupled to or adjacent to illuminator 1751, three branches 1761, 1762, 1763 curved towards a side surface of distal cap 1700, and a distal end 1755 at a distal front face 1798 of distal cap 1700. Various arrows are shown in FIG. 17 to show the direction that distal ends 1754, 1755 and branches 1761-1766 direct light from illuminators 1750, 1751. Light pipes 1752, 1753 may be made of any suitable clear or reflective material, such as injection-molded polycarbonate or polypropylene. By providing more than one outlet for light to travel from each illuminator 1750, 1751, the light produced by each illuminator 1750, 1751 may be more evenly distributed and may more effectively illuminate a patient's anatomy.

Any of the distal caps and other components described herein may be used to manufacture any of the other distal caps described herein, and other related distal caps or other components of a medical device.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a distal cap comprising:
an imaging device;
an illuminator;
a working channel;
a first material enveloping at least a portion of each of the imaging device, the illuminator, and the working channel; and
a second material enveloping the first material, wherein the second material is harder than the first material.

2. The medical device of claim 1, wherein the first material is molded onto the imaging device, the illuminator, and the working channel via injection molding.

3. The medical device of claim 2, wherein the second material is molded onto the first material via injection molding.

4. The medical device of claim 2, wherein the first material is completely covered by the second material.

5. The medical device of claim 1, wherein the distal cap further comprises a light pipe distal to the illuminator.

6. The medical device of claim 5, wherein the light pipe includes a plurality of branches extending to a side wall of the distal cap.

7. The medical device of claim 1, wherein the distal cap further comprises a printed circuit board operably coupled to the imaging device and the illuminator, wherein the printed circuit board is enveloped by the first material.

8. The medical device of claim 1, further comprising a shaft, wherein the distal cap is coupled to a distal end of the shaft.

9. The medical device of claim 8, further comprising a steering wire extending through the shaft, wherein a distal end of the steering wire is embedded in the first material and/or the second material of the distal cap.

10. The medical device of claim 1, wherein the second material extends circumferentially around the imaging device, the illuminator, and the working channel.

11. The medical device of claim 10, wherein a first distal front face of the imaging device, a second distal front face of the illuminator, and a third distal front face of the working channel are not in contact with the first material.

12. The medical device of claim 1, wherein the first material and/or the second material is transparent or translucent.

13. The medical device of claim 1, wherein the first material includes at least one first gate burr positioned proximal to the imaging device and the illuminator.

14. The medical device of claim 13, wherein the second material includes at least one second gate burr positioned at a proximal end of the second material.

15. The medical device of claim 1, wherein the second material extends through a channel of the first material, and wherein the second material abuts a distal front face of the illuminator.

16. A method for manufacturing a distal cap for a medical device, the method comprising:
positioning an imaging device, an illuminator, and a working channel in a mold;
introducing into the mold a first material;
introducing into the mold a second material different from the first material, wherein the second material sets to form the distal cap with the first material entirely enveloped by the second material; and
removing the distal cap from the mold.

17. The method of claim 16, wherein the imaging device and the illuminator are positioned within a cassette prior to positioning the imaging device, the illuminator, and the working channel in the mold.

18. The method of claim 16, further comprising:
stamping a distal end of a steering wire, wherein stamping the distal end changes a shape of the distal end; and
positioning the distal end of the steering wire within the mold prior to introducing the first material to the mold.

19. The method of claim 16, wherein positioning the imaging device, the illuminator, and the working channel in the mold includes positioning a plurality of imaging devices, a plurality of illuminators, and a plurality of working channels in the mold; and
wherein removing the distal cap from the mold includes removing a panel of distal caps from the mold.

20. A medical device comprising:
a shaft; and
a distal cap coupled to a distal end of the shaft, the distal cap comprising:
an imaging device;
an illuminator;
a working channel;
a first material enveloping at least a portion of the imaging device, the illuminator, and the working channel, wherein the first material is opaque; and
a second material enveloping the first material, wherein the second material is transparent and harder than the first material.
